(19) United States
(12) Patent Application Publication
(10) Pub. No.: US 2013/0281390 A1
(43) Pub. Date: Oct. 24, 2013

(54) METHOD FOR TREATING DRY EYE

(71) Applicant: Kurt E. Brubaker, Shady Cove, OR (US)

(72) Inventor: Kurt E. Brubaker, Shady Cove, OR (US)

(21) Appl. No.: 13/976,714

(22) PCT Filed: Dec. 28, 2011
(86) PCT No.: PCT/US11/67594

§ 371 Date: Jun. 27, 2013

Related U.S. Application Data
(60) Provisional application No. 61/427,959, filed on Dec. 29, 2010.

(51) Int. Cl. A61K 31/7052; A61K 31/232

(52) U.S. Cl. CPC A61K 31/7052; A61K 31/232
USPC 514/29

(57) ABSTRACT

This invention relates to methods of treating dry eye disease. The methods comprise identifying a patient suffering from dry eye disease, and topically administering to the eyes of the patient an effective amount of azithromycin and an effective amount of a retinoid. The method is useful in relieving the signs and symptoms of dry eye disease. This invention also relates to a pharmaceutical composition comprising azithromycin and a retinoid such as retinyl palmitate.

### TECHNICAL FIELD

This invention relates to methods of treating dry eye by administering azithromycin and a retinoid to a subject. The method is useful in relieving dry eye signs and symptoms. This invention also relates to a pharmaceutical composition comprising azithromycin and a retinoid such as retinyl palmitate.

### BACKGROUND OF THE INVENTION

Dry eye disease is a disorder due to an insufficient quantity of tears. The signs and symptoms of dry eye disease include ocular surface staining, eyelid swelling and redness, ocular irritation and foreign body sensation (gritty or sandy eyes). The quantity of tears can be reduced by either a failure to produce a sufficient amount of tears or by rapid evaporation of the tear film. Bron et al. (The Ocular Surface, 2: 149-164) disclose that the tear film lipid layer is the major barrier to evaporation from the ocular surface. A decrease in the thickness or functional integrity of the tear film may cause evaporative dry eye. Obstructive meibomian gland dysfunction is the most common cause of evaporative dry eye.

Dry eye disease is the general term for indications produced by abnormalities of the precorneal tear film characterized by a decrease in tear production or an increase in tear film evaporation, together with the ocular surface disease and symptoms that result. Approximately 38 million Americans are affected with some type of dry eye disorder. Among the indications that are referred to by the general term “dry eye disease” are: keratoconjunctivitis sicca (KCS), age-related dry eye, Stevens-Johnson syndrome, Sjogren's syndrome, ocular cicatrical pemphigoid, corneal injury, infection, Riley-Day syndrome, congenital alacrima, nutritional disorders or deficiencies (including vitamins), pharmacologic side effects, contact lens intolerance, eye stress and glandular and tissue destruction, environmental exposure to smog, smoke, excessively dry air, airborne particulates, autoimmune and other immunodeficient disorders, and comatose patients rendered unable to blink.

Dry eye disease, although seen pathologically during ophthalmic exams as superficial punctate keratopathy (SPK) of the ocular surface epithelium, is largely a symptomatic disease. Chronic dryness leads to pain and irritation that is often debilitating to the subject, preventing the performance of normal daily activities such as reading, driving, etc. Dry eye is most common in postmenopausal women; however, hormone replacement therapy has not been proven to help dry eye signs and symptoms.

Contact lens wear is commonly associated with symptoms of ocular irritation including symptoms such as dryness, discomfort, soreness, tiredness, and irritation, among other symptoms. Reports have shown that between 25-50% of contact lens wearers have these symptoms, albeit of differing frequencies and severities. Further, these symptoms have been shown to increase in their intensity or severity over the course of a day's wear of lenses, with an average increase of approximately 25% as demonstrated in at least one study. (Dougherty et al. Br. J. Ophthalmol. 1984;68:524-8)

Currently, the pharmaceutical treatment of dry eye disease is mostly limited to administration of artificial tears (saline solution) or anti-inflammatory agents (cyclosporine, steroids). Secretagogues (diquafosol, 15-HETE, rebamipide) to increase the production of tears are currently under development. In addition, artificial tears often have contraindications and incompatibility with soft contact lenses (M. Lemp, Cornea 9(1), S48-550 (1990)). The use of phosphodiesterase inhibitors, such as 3-isobutyl-1-methylxanthine (IBMX) to stimulate tear secretion is disclosed in U.S. Pat. No. 4,753,945. The effectiveness of these phosphodiesterase inhibitors has been investigated (J. Gilbard, et al., Arch. Ophthal, 112, 1614-16 (1994) and 109, 672-76 (1991); idem, Inv. Ophthal. Vis. Sci. 31, 1381-88 (1990)). Stimulation of tear secretion by topical application of melanocyte stimulating hormones is described in U.S. Pat. No. 4,868,154. Although these interventions can reduce inflammation and/or reduce SPK associated with dry eye, they have not been proven to significantly reduce the symptoms of dry eye.

Dry eye disease is different from blepharitis; the two diseases have different patient populations. Dry eye symptoms are dryness, burning, photophobia, foreign body sensation and grittiness in the eyes. Dry eye disease is characterized by an insufficient or defective tear film. The primary end points for studying dry eye diseases are corneal and conjunctival staining, measuring tear volume (Schirmer tests), Tear break-up time, dryness, burning, photophobia, foreign body sensation and grittiness.

Keratin is a tough, fibrous protein that is not water-soluble and is the main component of hair, nails and the epidermis (Ong, et al., Current Eye Research, 10, 1113-1119 (1991)). Keratinization is a process which involves the conversion of epithelial or other cell types in to cells that are largely comprised of keratin (Tseng, et al., Ophthalmology, 91, 545-552 (1984), and Jester, et al., Inv. Ophthal. Vis. Sci. 30, 927-935 (1989). Keratinization of cells in ocular surface tissues such as the conjunctiva, goblet cells and the meibomian glands may play a role in the development of dry eye (Nicholaides, et al., Inv, Ophthal. Vis. Sci. 30, 946-951 (1989). The secretions from the squamous epithelial cells and goblet cells in the conjunctiva contribute to the tear film mucin and aqueous components. Overexpression of keratin, or hyperkeratinization, of these cell types may impact both the quality and quantity of the tear film. Keratinization of the meibomian glands may occur on the lid margin, obstructing the orifice of the glands, or inside the glands to the epithelial lining of the glands. Hyperkeratinization either in the glands or of the surface epithelium may alter the lipid secretions which comprise the outer layer of the tear film.

Retinoids include Vitamin A (retinol), retinoic acid, and retinyl palmitate as well as related compounds that are synthetic or naturally occurring cellular components or metabolites. The effects of RA and synthetic derivatives are mediated by two classes of nuclear receptors, the retinoic acid receptors which belong to the erbA-related steroid/thyroid nuclear receptor superfamily and the retinoid X receptors which also belong to the same super family of steroid/thyroid hormones (Gorodeski, et al., Am. J. Physiol. Cell. Physiol. 275, 758-765 (1998).

Vitamin A and related retinoids are involved in the maintenance of mucosal membranes via control of the proliferation and differentiation of epithelial cells. A deficiency of retinoids results in a gradual change of the ocular mucosa to a non-secretory keratinized epithelium. (Kobayashi, et al., Ophthalmologica, 211, 358-361 (1997)). Retinoic acid plays a fundamental role in cell proliferation, and cell differentiation and it may also prevent malignant transformation (Darmon, 1991, Sem. Dev. Biol. 2:219).

Retinoids have been utilized to treat a number of conditions involving keratinization of epithelial tissue, including: acne vulgaris, psoriasis, wound healing and premalignant lesions (Kligman, A., Cutis, 39, 486-488 (1987). Formulations containing retinoids have also been utilized to treat ocular disorders involving the epithelium, such as dry eye, Stevens-Johnson syndrome (Kobayashi, et al., Ophthalmologica, 211, 358-361 (1997); Selek, et al., Eur. J. Ophthalmol, 10, 121-127 (2000) and Kim, et al., Amer. J. Oph, 147, 206-213.e3 (2009)). Topical retinoid formulations include ointments and liquid formulations that may be applied 2-4 times per day for one or more months. Increases in goblet cell density in ocular mucosal tissue, tear break up time and Schirmer score measurements have been noted following topical retinoid therapy.

Systemic treatment with retinoids for epithelial keratinization disorders may be effective, but side effects due to toxicity are common (Kligman, A., Cutis, 39, 486-488 (1987). Topical preparations allow for sufficient efficacy at the site of the disorder, yet avoid systemic side effects. Side effects of topical retinoid formulations include irritation and redness during the initial period of therapy. Practices to reduce the side effects of topical retinoid formulations include a reduction in the concentration of the active ingredient, a reduction in frequency of administration or discontinuation of therapy, which make the retinoid treatment less effective.

Azithromycin is a macrolide antibiotic. AZASITE® (azithromycin ophthalmic solution) is a 1% sterile aqueous topical ophthalmic solution of azithromycin formulated in DURASITE® (polycarbophil, edetate disodium, sodium chloride). AZASITE® is approved by the U.S. Food and Drug Administration (FDA) for treatment of bacterial conjunctivitis, caused by susceptible isolates of CDC coryneform group G, Haemophilus influenzae,

Staphylococcus aureus, Streptococcus mitis group, and Streptococcus pneumoniae (AZASITE® Package Insert, 2007). The recommended dosage regimen for the treatment of bacterial conjunctivitis is as follows: instill 1 drop in the affected eye(s) twice daily, 8 to 12 hours apart for the first 2 days and then instill 1 drop in the affected eye(s) once daily for the next 5 days (AZASITE® Package Insert, 2007).

Azithromycin has immunomodulatory and anti-inflammatory effects that are separate from the antimicrobial effects. Studies have been conducted that demonstrate a reduction in inflammatory cell influx, pro-inflammatory mediator release (e.g. cytokines and chemokines), and tissue remodeling mediators such as matrix metalloproteinases (Amsden, G. W., Journal of Antimicrobial Chemotherapy, 55, 10-21 (2005), Li, D. Q., Invest Ophthalmol Vis Sci, 51(11), 5623-9 (2010)).

Topical application of azithromycin to the ocular surface has been shown to improve the quality of the meibomian gland secretions. Daily administration of topical azithromycin in subjects with meibomian gland dysfunction improved the physicochemical properties of the meibum towards that of normal subjects (Foulks, G. N., Cornea, 29(7), 781-8 (2010)).

Despite the high prevalence of dry eye disease, present therapies often result in poor patient compliance and disappointing results. Therefore, there is a need for an effective and safe method to treat dry eye disease.

### SUMMARY OF THE INVENTION

The present invention is directed to a method for treating dry eye disease. The method comprises the steps of: identifying a patient suffering from dry eye disease, and topically administering to the patient an effective amount of azithromycin or a pharmaceutically acceptable salt thereof and an effective amount of a retinoid. Azithromycin and the retinoid can be administered either sequentially or by co-administration. Preferred azithromycin is azithromycin monohydrate and preferred retinoid is retinyl palmitate. An effective concentration of azithromycin is about 0.5%-2% (w/v), and an effective concentration of retinyl palmitate is about 0.01-0.1% (w/v). An effect dosing regimen would consist of topical administration of 1-2 drops (approximately 50 microliters) in each eye once or twice a day.

The present invention is also directed to a pharmaceutical composition comprising active ingredients and a pharmaceutically acceptable carrier, wherein the active ingredients consisting essentially of 0.5-2% (w/v) of azithromycin and 0.01-1% (w/v) retinyl palmitate. The pharmaceutical composition preferably is an ophthalmic solution or suspension.

### DETAILED DESCRIPTION OF THE INVENTION

The inventor has discovered an effective method for treating dry eye disease by administering azithromycin and a retinoid to a patient suffering from dry eye disease. The inventor has discovered that combined administration of azithromycin and a retinoid is more effective in treating dry eye disease than the single administration of either azithromycin or a retinoid.

The present invention is directed to a method for treating dry eye. The method comprises the steps of: identifying a patient suffering from dry eye disease, and topically administering to the patient an effective amount of azithromycin or a pharmaceutically acceptable salt thereof and an effective amount of a retinoid. Azithromycin and the retinoid can be administered to the patient separately or co-administered in one single formulation.

In another embodiment, the method comprises the steps of: identifying a patient suffering from dry eye, and administering to the patient a pharmaceutical composition comprising active ingredients and a pharmaceutically acceptable carrier, wherein the active ingredients consisting essentially of an effective amount of azithromycin and an effective amount of a retinoid. The active ingredients only include azithromycin and a retinoid and those that do not materially affect the basic and novel characteristics of the claimed invention.

Particularly, the present invention is suitable for treating dry eye diseases caused by one or more of keratoconjunctivitis sicca, age-related dry eye, Stevens-Johnson syndrome, Sjogren's syndrome, ocular cicatrical pemphigoid, corneal injury, infection, Riley-Day syndrome, congenital alacrima, nutritional disorders or deficiencies, pharmacologic side effects, contact lens intolerance, eye stress resulting in glandular and tissue destruction, autoimmune disorders, immuno-deficient disorders, comatose patients who are unable to blink, or environmental exposure to smog, smoke, excessively dry air, or airborne particulates. The present invention is also suitable to treat patients suffering from dry eye disease, but not suffering from blepharitis.

The present invention is also directed to a method for reducing contact lens intolerance in a subject. The method comprises the steps of: identifying a subject suffering from contact lens intolerance due to dry eye disease; and topically administering to the patient a pharmaceutical composition comprising active ingredients and a pharmaceutically acceptable carrier, wherein the active ingredients consisting essentially of an effective amount of azithromycin and an effective amount of a retinoid. The active ingredients only include azithromycin and a retinoid and those that do not materially affect the basic and novel characteristics of the claimed invention. The present method increases comfortable contact lens wearing time and/or total duration of contact lens wearing time per day in the subject.

“Retinoids” as used in this application, refer to a class of compounds consisting of four isoprenoid units joined in a head-to-tail manner. All retinoids may be formally derived from a monocyclic parent compound containing five carbon-carbon double bonds and a functional group at the terminus of the acyclic portion. Retinoids are a class of chemical compounds that are chemically related to vitamin A (retinol). Examples of retinoids useful in the present invention include: vitamin A (retinol), retinyl palmitate, retinal, tretinoin (retinoic acid), isotretinoin, etretinate, acitretin, tazarotene, bexarotene, and adapalene. Retinyl palmitate is a preferred compound for this invention.

“An effective amount” as used herein, is meant an amount that has a therapeutic effect, which reduces the signs and/or symptoms of dry eye disease. In the present method, the effective concentration of azithromycin is about 0.5%-2% (w/v), e.g., about 1%, and an effective concentration of retinyl palmitate is about 0.01-0.1% (w/v), e.g., about 0.05%. An effect dosing regimen would consist of topical administration of 1-2 drops (approximately 50 microliters) in each eye once or twice a day. In one embodiment, the effective concentration of azithromycin is 1% (w/v), and an effective amount of retinyl palmitate is 0.05% (w/v).

“About” as used in this application, refers to ±10% of the recited value.

### Pharmaceutical Composition

The present invention is directed to a pharmaceutical composition comprising azithromycin or a pharmaceutically acceptable salt thereof, a retinoid, and a pharmaceutically acceptable carrier. Preferred azithromycin is azithromycin monohydrate and preferred retinoid is retinyl palmitate. The pharmaceutical composition preferably is an ophthalmic solution or suspension. A preferred pharmaceutical composition comprises active ingredients and a pharmaceutically acceptable carrier, wherein the active ingredients consisting essentially of 0.5-2% of azithromycin and 0.01-1% retinyl palmitate.

The topical solution containing azithromycin and a retinoid can contain a physiologically compatible vehicle, as those skilled in the ophthalmic art can select using conventional criteria. The ophthalmic vehicles include, but are not limited to, saline solution, water polyethers such as polyethylene glycol, polyvinyls such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate and salts such as sodium chloride and potassium chloride.

Preferred ophthalmic formulations of azithromycin and retinoids suitable for the present method are those disclosed in U.S. Pat. Nos. 6,239,113, 6,569,443 and 7,056,893; the formulations of which are incorporated herein by reference. For example, the formulation is an aqueous polymeric suspension comprising water, azithromycin, retinyl palmitate, and 0.1 to 10% of a polymeric suspending agent. The polymeric suspending agent comprises a water-swellable water-insoluble crosslinked carboxy-vinyl polymer. For example, the polymeric suspending agent comprises least 90% (w/v) acrylic acid monomers and 0.1% to 5% (w/v) crosslinking agent. The formulations may further comprise polyvinyl alcohols and providone.

AZASITE® (azithromycin ophthalmic solution), which is a 1% (w/v) sterile aqueous topical ophthalmic solution of azithromycin formulated in DURASITE® (polycarbophil, edetate disodium, sodium chloride), is a preferred ophthalmic formulation. Retinyl palmitate can be added to AZASITE® to form a combination formulation. The preferred ophthalmic formulations are able to keep prolonged high azithromycin and retinyl palmitate concentrations on the ocular surface, thus facilitating its penetration into the eye tissues.

The formulation optionally includes a preservative, such as benzalkonium chloride and other inactive ingredients such as EDTA. For a short term use of less than two weeks, preferably less than one week, benzalkonium chloride has the benefit of increasing the penetration of azithromycin into eye tissues. However, for chronic (over two weeks) use, preferred formulations are those without any preservatives due to the potential for damage to the corneal epithelium that may result from long term, frequent exposure to preservatives such as benzalkonium chloride. The formulations without preservatives are prepared in a unit dose and stored in a single-use container.

The pH of the formulation is typically adjusted by adding any physiologically and ophthamologically acceptable pH adjusting acids, bases or buffers to within the range of about 5 to 7.5; preferably 6 to 7. Examples of acids include acetic, boric, citric, lactic, phosphoric, hydrochloric, and the like, and examples of bases include sodium hydroxide, sodium phosphate, sodium borate, sodium citrate, sodium acetate, sodium lactate, tromethamine, THAM (trishydroxymethylamino-methane), and the like. Salts and buffers include citrate/dextrose, sodium bicarbonate, ammonium chloride and mixtures of the aforementioned acids and bases.

The osmotic pressure of the aqueous ophthalmic composition is generally from about 200 to about 400 milliosmolar (mOsM), more preferably from 260 to 340 mOsM. The osmotic pressure can be adjusted by using appropriate amounts of physiologically and ophthamologically acceptable ionic or non-ionic agents. Sodium chloride is a preferred ionic agent, and the amount of sodium chloride ranges from about 0.01% to about 1% (w/v), and preferably from about 0.05% to about 0.45% (w/v). Equivalent amounts of one or more salts made up of cations such as potassium, ammonium and the like and anions such as chloride, citrate, ascorbate, borate, phosphate, bicarbonate, sulfate, thiosulfate, bisulfate, sodium bisulfate, ammonium sulfate, and the like can be used in addition to or instead of sodium chloride to achieve osmolality within the above-stated range. Further, non-ionic agents such as mannitol, dextrose, sorbitol, glucose and the like can also be used to adjust the osmolality.

### Administration

The inventor has also discovered that topical administration is an effective method for delivering azithromycin and the retinoid. Instillation to the ocular surface is a localized administration method and can therefore be more effective in reaching the target area, i.e., the eye, and providing a high and localized concentration of azithromycin and the retinoid. Topical instillation avoids undesired side effects due to systemic exposure of the azithromycin and the retinoid and reduces the risk of patients from developing antibiotic resistance.

Formulations described by the present invention can be administered to the eyes of a patient by any suitable means, but are preferably administered as a liquid or gel suspension in the form of drops, spray or gel. In one embodiment, the formulation is in the form of drops, and is dropped onto the ocular surface. In another embodiment, the formulation is contained within a swab or sponge which can be applied to the ocular surface. In another embodiment, the formulation is contained within a liquid spray or ointment which can be applied to the ocular surface. In another embodiment, the formulation is injected directly into the lacrimal tissues or onto the eye surface. In a further embodiment, the formulation (e.g., in the form of drops) is first applied on a finger tip or other applicator, then applied or rubbed directly onto the lid margin. Alternatively, azithromycin and a retinoid can be applied to the eye via liposomes. Further, azithromycin and a retinoid can be infused into the tear film via a pump-catheter system. Another embodiment of the present invention involves azithromycin and a retinoid contained within a continuous or selective-release device, for example, membranes such as, but not limited to, those employed in the OCUSERT™ System (polymeric ocular inserts for the administration of drugs, Alza Corp., Palo Alto, Calif.). As an additional embodiment, azithromycin and a retinoid can be contained within, carried by, or attached to contact lenses or other compatible controlled release materials, which are placed on the eye.

In one embodiment, the azithromycin and retinoid are administered 1, 2, 3 or 4 times a day sequentially (i.e. one after the other), or co-administered together. In one embodiment, azithromycin and retinyl palmitate are admixed as one pharmaceutical composition and administered to patients by instillation on to the ocular surface. One single pharmaceutical composition and one single treatment provide ease of use and result in better compliance of patients. In another embodiment, azithromycin and retinyl palmitate are each in a separate formulation and administered separately to patients.

The daily dose to treat dry eye disease can be divided among one or several unit dose administrations. The daily dose, for example, can range from one drop (about 50 μl), one to four times a day, depending upon the age and condition of the subject. A preferred regimen is one drop of a 1% azithromycin (w/v) and 0.05% (w/v) retinyl palmitate solution, about 1 to 2 times a day. For example, a preferred dosage is one drop in each eye twice a day for two days and then once a day thereafter.

When treating dry eye disease, the present method can be combined with mechanical therapy such as warm compress or lid hygiene (lid cleansing).

### Unexpected Advantages of the Present Invention

The inventor has discovered that the combined administration of azithromycin and a retinoid to a dry eye disease patient has several advantages that cannot be achieved by a single administration of either azithromycin or a retinoid.

Dry eye disease is a chronic disease, and subjects with dry eye disease often have physical alterations to the meibomian glands and the epithelium surrounding the orifices of the meibomian glands, as well as the corneal and conjunctival epithelium. The inventor has discovered that the combined treatment of a retinoid and azithromycin allows for a normal meibomian gland function which improves the quality of the meibomian gland secretions, and allows for the improved secretions to be secreted and reach the ocular surface. The combined treatment also provides enhanced mucosal secretions from the ocular surface epithelium. Additionally, the anti-inflammatory properties of azithromycin reduce the side effects of irritation and inflammation during the early phase of a retinoid treatment, which enhance the tolerability of topically applied retinoid without having to reduce the amount or frequency of the retinoid application.

The invention is illustrated further by the following examples which are not to be construed as limiting the invention in scope or spirit to the specific procedures described in it.

### EXAMPLES

### Example 1

### Use of Azithromycin and a Retinoid for Reducing Symptoms in Patients with Dry Eye Disease

### Objectives

The objective of this study is to compare the efficacy of an ophthalmic formulation of azithromycin 1% and retinyl palmitate 0.05% combined, versus the ophthalmic formulations of the respective active ingredients alone over a four week treatment period on the symptoms of subjects with dry eye disease.

### Subjects

Subjects are 18 years of age or older, and have a clinical diagnosis of mild to moderate dry eye disease. A total of 120 subjects are enrolled in the study.

### Methods

This is a double-masked study. At Visit 1 (Day 1), all subjects are randomized in 1:1:1 ratio to receive either (a) combination of azithromycin and retinyl palmitate or (b) azithromycin alone or (c) retinyl palmitate alone, for 30 days. Study drug is administered as one drop in each eye BID for the first 2 days and then QD for the remainder of the study. Study drugs are self-administered by the subjects. The subjects are prohibited from using any ocular or other medications that could confound the results of the assessments during study participation, such as antihistamines, steroids, antibiotics or preserved artificial tears.

Patients return for Visit 2 (Day 14), and Visit 3 (Day 30) and are asked to rate their symptoms including: ocular itching, ocular burning/pain, and foreign body sensation.

### Scores on the Symptoms of Dry Eye Disease

Patients rate the severity of their dry eye symptoms at Visits 1, 2, and 3 according to the following three classifications.

### Eyelid itching

Do your eyelids feel itchy?
(0) None: My eyelids do not feel itchy.
(1) Mild: Once in a while, my eyelids feel slightly itchy, but I do not have a desire to rub them.
(2) Moderate: Occasionally, my eyelids feel itchy, and I need to rub them.
(3) Severe: It is difficult to relieve the sensation of itchiness even when I rub my eyelids.
(4) Very severe: I have unbearable eyelid itching with an irresistible urge to rub my eyelids.

### Foreign Body Sensation/Sandiness, Grittiness

Do you feel like there's something sandy or gritty in your eye?
(0) None: My eyes do not feel sandy or gritty.
(1) Mild: I am aware of the surface of my eyes once in a while.
(2) Moderate: My eyes feel like there is something small in them occasionally.
(3) Severe: My eyes feel like there is something large or gritty in them.
(4) Very severe: I am unable to open my eyes due to feeling of a foreign body in my eyes.

### Ocular Burning or Pain

Are your eyes burning or painful?
(0) None: My eyes do not burn or ache.
(1) Mild: I am aware of the surface of my eyes; they mildly burn or ache.
(2) Moderate: I feel my eyes are burning, but still tolerable
(3) Severe: My eyes feel throbbing or fiery due to burning/pain.
(4) Very severe: I am unable to open my eyes due to burning/pain

### Results

The mean scores for individual symptoms for each group are compared for Visits 2-3 to baseline (Visit 1). A statistically significant difference (p<0.05) is observed in favor of the azithromycin and retinyl palmitate treatment group for at least one of the Visits.

### Conclusions

The above results indicate that combination of azithromycin and retinyl palmitate reduces the symptoms of dry eye disease significantly greater than either azithromycin or retinyl palmitate alone.

### Example 2

### Use of Azithromycin and a Retinoid for Treating Patients with Contact Lens Related Dry Eye Disease

### Objectives

The objective of this study is to compare the efficacy of the study drugs (1% Azithromycin in an ophthalmic solution and 0.05% retinyl palmitate in an ophthalmic solution), versus rewetting drops (Visine for Contacts™) over a four week treatment period in patients with contact lens-realted dry eye disease (CLDE).

### Subjects

Subjects are 18 years of age or older. Subjects have a positive diagnosis of CLDE based on their responses to the Contact Lens Dry Eye Questionnaire (CLDEQ, Nichols et al, Cornea 21:469-75 (2002)) and a score of ≧2, (i.e., moderate severity on a 0-4 scale, where 0=no ocular dryness and 4=very severe ocular dryness) on a self-reported assessment of ocular dryness at the screening Visit. Over 30 subjects are enrolled in the study.

### Methods

### Visit 1 (Day 1)

Subjects are asked to rate their level of ocular dryness on a 0-4 scale. Tear osmolarity is then assessed for each eye using the TearLab Osmolarity System®. Subjects are then instructed to remove their contact lenses and slit-lamp biomicroscopy and external eye exams are performed. Fluorescein corneal staining is then performed on each eye (5 μl of 2% fluorescein applied to the ocular surface) and graded according to National Eye Institute (NEI) criteria.

Eligible subjects are then randomized to receive either the study drugs (1% Azithromycin and 0.05% retinyl palmitate), or rewetting drops (Visine for Contacts™) in a 1:1 ratio and they are not masked as to the treatment assignment. Subjects randomized to azithromcyin and retinyl palmitate are instructed to administer one drop to each eye BID for both drugs the first two days and then one drop QID for both drugs for days 3-29. Subjects randomized to rewetting drops are instructed to administer one to two drops QID throughout the 29 day treatment period. The first dose of study medication is self-administered at the clinic once dosing instructions are provided. Subjects are instructed to apply a new pair of contact lenses to their eyes 10 minutes after the instillation of study medication. Subjects are queried for adverse events (AEs) and scheduled to return for Visit 2 in approximately two weeks.

Subjects are instructed to record the AM ocular dryness assessment prior to instilling study medication (and lens application), and record PM dryness levels prior to lens removal each day. Further, subjects are required to record the time that contacts are inserted, the time lenses became uncomfortable and the time lenses are removed.

### Visit 2 (Day 14±2)

Concomitant medication usage is recorded, an AE query performed, study medication and diary cards are collected. Subjects undergo habitual HCVA and LCVA assessment, complete the CLDEQ, and have tear osmolarity readings measured prior to removing their contact lenses. Biomicroscopy, external eye exam, and fluorescein corneal staining are subsequently performed. The subjects then re-apply their lenses and additional study medication and diary cards are provided.

### Visit 3 (Day 29±1)

All assessments and procedures are performed the same manner as at Visit 2.

### Statistical Analyses

The primary efficacy endpoint for this study is the change from baseline in the duration of subject-reported comfortable contact lens daily wear time (hours/day) at the Week 4 Endpoint. The Week 4 Endpoint is defined as the average of the change from baseline values for comfortable contact lens daily wear time from days 23 to 29.

Secondary efficacy endpoints from the data captured from the diaries included: change from baseline in comfortable contact lens wear time at Week 1 (days 2-8), Week 2 (days 9-15), Week 3 (days 16-22), change from baseline in total contact lens wear time (Week 1-4 time intervals as defined for comfortable contact lens wear), and change from baseline in subject-reported levels of ocular dryness both at the morning (AM) assessment and evening (PM) assessment.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude this specification.

1-12. (canceled)

13. A method for treating dry eye disease, comprising the steps of:
a. identifying a patient suffering from dry eye disease; and
b. topically administering to the eyes of said identified patient an effective amount of azithromycin and an effective amount of a retinoid.

14. The method according to claim 13, wherein said azithromycin and said retinoid are administered separately.

15. The method according to claim 13, wherein said azithromycin and said retinoid are co-administered in one composition.

16. The method according to claim 13, wherein said azithromycin is azithromycin monohydrate.

17. The method according to claim 13, wherein the effective amount of azithromycin is about 0.5-2% (w/v).

18. The method according to claim 13, wherein the effective amount of azithromycin is about 1% (w/v).

19. The method according to claim 13, wherein said retinoid is selected from the group consisting of retinol, retinyl palmitate, tretinoin, isotretinoin, etretinate, acitretin, tazarotene, bexarotene, and adapalene.

20. The method according to claim 19, wherein said retinoid is retinyl palmitate.

21. The method according to claim 20, wherein the effective amount of retinyl palmitate is about 0.01-1% (w/v).

22. The method according to claim 20, wherein the effective amount of retinyl palmitate is about 0.05% (w/v).

23. A method for treating dry eye disease, comprising the steps of:
a. identifying a patient suffering from dry eye disease; and
b. topically administering to the eyes of said identified patient a pharmaceutical composition comprising active ingredients and a pharmaceutically acceptable carrier, wherein the active ingredients comprise an effective amount of azithromycin and an effective amount of a retinoid.

24. The method according to claim 23, wherein said azithromycin is azithromycin monohydrate.

25. The method according to claim 23, wherein the effective amount of azithromycin is about 0.5-2% (w/v).

26. The method according to claim 23, wherein the effective amount of azithromycin is about 1% (w/v).

27. The method according to claim 23, wherein said retinoid is selected from the group consisting of retinol, retinyl palmitate, tretinoin, isotretinoin, etretinate, acitretin, tazarotene, bexarotene, and adapalene.

28. The method according to claim 27, wherein said retinoid is retinyl palmitate.

29. The method according to claim 28, wherein the effective amount of retinyl palmitate is about 0.01-1% (w/v).

30. The method according to claim 28, wherein the effective amount of retinyl palmitate is about 0.05% (w/v).

31. A pharmaceutical composition for treating dry eye disease comprising active ingredients and a pharmaceutically acceptable carrier, wherein the active ingredients consist essentially of 0.5-2% (w/v) of azithromycin and 0.01-1% (w/v) retinyl palmitate.
